(19) United States
(12) Patent Application Publication
(10) Pub. No.: US 2026/0199629 A1
(43) Pub. Date: Jul. 16, 2026

(54) DIGITAL THERAPY PROCESSING SYSTEM AND METHOD BASED ON MULTIMODAL AND INTELLIGENT MONITORING

(71) Applicant: Wenzhou Medical University, Wenzhou (CN)

(72) Inventors: Wenjing Yan, Wenzhou (CN); Ruofei Xu, Wenzhou (CN); Heng Miao, Wenzhou (CN); Qiannan Ruan, Wenzhou (CN); Haoyu Huang, Wenzhou (CN)

(21) Appl. No.: 19/269,075

(22) Filed: Jul. 15, 2025

(30) Foreign Application Priority Data
Jan. 10, 2025 (CN) 2025100381764

(51) Int. Cl. A61M 21/02; A61M 21/00

(52) U.S. Cl. CPC A61M 21/02; A61M 2021/0022; A61M 2021/0027; A61M 2021/0044; A61M 2230/06; A61M 2230/65

(57) ABSTRACT

A digital therapy processing system based on multimodal and intelligent monitoring applied in an intelligent terminal includes a data acquisition module configured to obtain self-assessment information input by a user, a setting module configured to set bilateral stimulation parameters based on the self-assessment information, a multimodal bilateral stimulation module configured to provide a comprehensive bilateral stimulation experience based on the bilateral stimulation parameters, a monitoring module configured to perform real-time emotional state monitoring based on obtained monitoring data acquired through an external intelligent device during system operation to obtain a real-time monitored emotional state, and a self-adjustment module configured to automatically adjust the bilateral stimulation parameters based on the real-time monitored emotional state and a preset adjustment strategy, thereby achieving adaptive adjustment of a personalized solution. The system and method reduce dependence on professional therapists, achieves self-adaptive adjustment of personalized solution, and ensures safety and effectiveness of the entire process.

### CROSS-REFERENCE TO RELATED APPLICATION

This application claims priority to Chinese patent application No. CN202510038176.4, filed to China National Intellectual Property Administration (CNIPA) on Jan. 10, 2025, which is herein incorporated by reference in its entirety.

### TECHNICAL FIELD

The disclosure relates to the technical field of medical technologies, and particularly to a digital therapy processing system and a method based on multimodal and intelligent monitoring.

### BACKGROUND

Eye movement desensitization and reprocessing (EMDR) is a psychotherapeutic technique used to treat post-traumatic stress disorder (PTSD). EMDR helps patients reprocess traumatic memories and alleviate associated symptoms by guiding them to recall the traumatic memories while simultaneously engaging in bilateral stimulation (such as eye movements, tactile, or auditory stimulation).

Currently, EMDR therapy is primarily conducted by professional therapists in clinics. Therapists typically use visual stimulation such as moving fingers or light bars. For example, Shapiro (1989) described the initial EMDR scheme in her research, which uses the therapist's moving fingers as the visual stimulation.

It can be seen that the technologies mentioned above have the following main drawbacks.
(1) Highly dependent on professional therapists, which limits the accessibility of treatment.
(2) Lack of real-time emotion monitoring, which may pose security risks.
(3) The use of single-modality stimulation may limit the therapeutic effect.
(4) It is difficult to achieve self-administered treatment.
(5) Lack of personalized and adaptive adjustment mechanisms.

### SUMMARY

To solve the deficiencies in the related art, the embodiments of the disclosure provide a digital therapy processing system and a method based on multimodal and intelligent monitoring to solve the technical problems existing in the related art.

In an embodiment, a digital therapy processing system based on multimodal and intelligent monitoring applied in an intelligent terminal is provided, and the digital therapy processing system includes a data acquisition module, a setting module, a multimodal bilateral stimulation module, a monitoring module, and a self-adjustment module. The data acquisition module is configured to obtain self-assessment information input by a user. The setting module is configured to set initial bilateral stimulation parameters based on the self-assessment information. The multimodal bilateral stimulation module is configured to provide a comprehensive bilateral stimulation experience for the user based on the initial bilateral stimulation parameters, and the bilateral stimulation experience includes visual stimulation, tactile stimulation, and auditory stimulation. The monitoring module is configured to perform real-time emotional state monitoring based on obtained monitoring data during system operation to obtain a real-time monitored emotional state of the user, and the monitoring data is acquired through an external intelligent device. The self-adjustment module is configured to automatically adjust the initial bilateral stimulation parameters based on the real-time monitored emotional state and a preset adjustment strategy, thereby achieving adaptive adjustment of a personalized solution.

In an embodiment, the digital therapy processing system further includes a storage module, and the storage module is configured to record data during a therapeutic process, and configured to encrypt storage and access control to ensure data security.

In an embodiment, each of the visual, the tactile stimulation, and the auditory stimulation includes multiple sets of stimulation, and an interval time is preset between the visual stimulation and the tactile stimulation, between the visual stimulation and the auditory stimulation, and between the tactile stimulation and the auditory stimulation.

In an embodiment, for each of the visual stimulation, the tactile stimulation, and the auditory stimulation, an execution content of each of the multiple sets of stimulation is different from one another.

In an embodiment, the digital therapy processing system further includes a baseline module, the baseline module is configured to perform a baseline test on the user before providing the comprehensive bilateral stimulation experience, to obtain personalized baseline values, and transmit the personalized baseline values to the self-adjustment module as a reference for subsequent automatic parameter adjustment.

In an embodiment, the automatically adjust the initial bilateral stimulation parameters based on the real-time monitored emotional state and a preset adjustment strategy includes:
when the monitoring data of the user is detected to be below a first threshold of the personalized baseline values of the user, indicating the user being in a highly stress state; in response to the user being in the highly stress state, automatically reducing the initial bilateral stimulation parameters to lower an intensity of stimulation; and shortening a duration of a single therapy simultaneously;
when the monitoring data of the user is detected to rapidly rise and exceed a second threshold of the personalized baseline values of the user, indicating the user being in a highly tense state; in response to the user being in the highly tense state, automatically adjusting the initial bilateral stimulation parameters to first suspend existing visual stimulation, and switching to the auditory stimulation or the tactile stimulation.

In an embodiment, a digital therapy processing method based on multimodal and intelligent monitoring applied to the digital therapy processing system mentioned above, which includes steps as follows: obtaining self-assessment information input by a user; setting initial bilateral stimulation parameters based on the self-assessment information; providing a comprehensive bilateral stimulation experience for the user based on the initial bilateral stimulation parameters, where the bilateral stimulation experience includes visual stimulation, tactile stimulation, and auditory stimulation; performing a real-time emotional state of the user monitoring based on obtained monitoring data during system operation, where the monitoring data is acquired through an external intelligent device; and automatically adjusting the initial bilateral stimulation parameters based on the real-time monitored emotional state and a preset adjustment strategy, thereby achieving adaptive adjustment of a personalized solution.

In an embodiment, each of the data acquisition module, the setting module, the multimodal bilateral stimulation module, the monitoring module, the self-adjustment module, the storage module, and the baseline module is embodied by at least one processor and at least one memory coupled to the at least one processor, and the at least one memory stores computer programs executable by the at least one processor.

The beneficial effects of the disclosure are as follow.

The digital therapy processing system based on multimodal and intelligent monitoring of the disclosure combines EMDR with multimodal and intelligent monitoring. When initiating multimodal bilateral stimulation, the digital therapy processing system conducts real-time emotion monitoring at the same time. Based on the monitoring results (i.e., real-time monitored emotional state), the digital therapy processing system automatically adjusts the initial bilateral stimulation parameters for adaptive adjustment of the personalized solution, to ensure safety and effectiveness of the entire process, thereby reducing reliance on professional therapists, enhancing EMDR accessibility. Therapy effectiveness is enhanced through multimodal stimulation, to provide a more comprehensive sensory experience. The real-time emotional state monitoring is used to make timely adjustments to ensure the safety of treatment. Its automated operation has improved convenience and is suitable for home-based self-treatment, and it can perform adaptive adjustments of the personalized solution based on monitoring conditions to meet the usage needs of different users.

The digital therapy processing method based on multimodal and intelligent monitoring in the disclosure shares the same inventive concept as digital therapy processing system mentioned above. It also offers the same beneficial effects.

### BRIEF DESCRIPTION OF DRAWINGS

In order to provide a clearer explanation of the specific embodiments of the disclosure or the technical solutions in the related art, the following will briefly introduce the attached drawings required for the specific embodiments or related art description. In the attached drawings, similar elements or parts are generally identified by similar reference numerals. In the attached drawings, each component or part may not be drawn in actual proportion.

FIG. 1 illustrates a schematic structural block diagram of a digital therapy processing system based on multimodal and intelligent monitoring of the disclosure.

FIG. 2 illustrates a flowchart of a digital therapy processing method based on the multimodal and intelligent monitoring in the disclosure.

### DETAILED DESCRIPTION OF EMBODIMENTS

Below, the technical solutions in the embodiments of the present disclosure will be clearly and completely described in conjunction with the attached drawings. Apparently, the described embodiments are a part of the embodiments of the disclosure, not all of them. Based on the embodiments of the disclosure, all other embodiments obtained by those skilled in the art without creative labor are within the scope of protection of the disclosure.

It should be understood that, when used in the specification and the appending claims, the terms “include” and “contain” indicate the presence of the described features, entirety, steps, operations, elements, and/or components, but do not exclude the existence or addition of one or more other features, entirety, steps, operations, elements, components, and/or their combinations.

It should also be understood that the terms used in the specification of the disclosure are merely for describing specific embodiments and are not intended to limit the disclosure. As used in the specification and the appending claims, unless the context clearly indicates otherwise, the singular forms “a”, “an”, and “the” are intended to include the plural forms.

It should further be understood that the term “and/or” used in the specification and the appending claims refers to any combination of one or more of the items listed in association, as well as all possible combinations thereof, and includes these combinations.

As used in the specification and the appending claims, the term “if” may be interpreted, depending on the context, as “when . . . ,” “once,” “in response to determining,” or “in response to detecting.” Similarly, the phrases “if determined” or “if (the described condition or event) is detected” may be interpreted, depending on the context, as “once determined,” “in response to determining,” “once (the described condition or event) is detected,” or “in response to detecting (the described condition or event).”

It should be noted that, unless otherwise specified, the technical or scientific terms used in the disclosure should be understood in their ordinary sense by those skilled in the art to which the disclosure belongs.

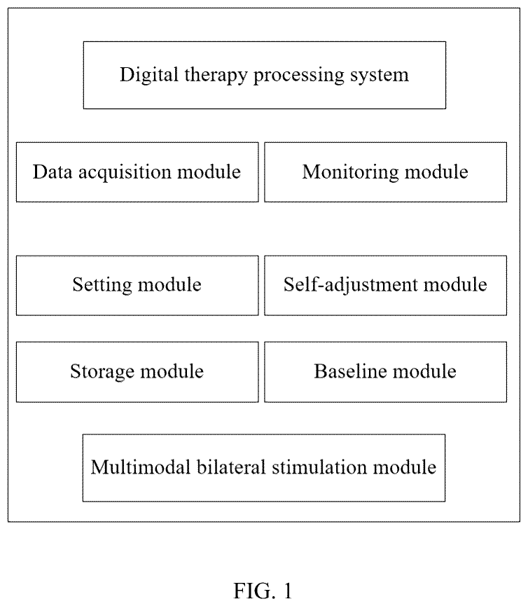

As shown in FIG. 1, a schematic structural block diagram of a digital therapy processing system based on multimodal and intelligent monitoring applied in an intelligent terminal is illustrated. The digital therapy processing system includes a data acquisition module, a setting module, a multimodal bilateral stimulation module, a monitoring module, and a self-adjustment module.

The data acquisition module is configured to obtain self-assessment information input by a user. The setting module is configured to set initial bilateral stimulation parameters based on the self-assessment information. The multimodal bilateral stimulation module is configured to provide a comprehensive bilateral stimulation experience for the user based on the initial bilateral stimulation parameters, and the bilateral stimulation experience includes visual stimulation, tactile stimulation, and auditory stimulation. The monitoring module is configured to perform real-time emotional state monitoring based on obtained monitoring data during system operation to obtain a real-time monitored emotional state of the user, and the monitoring data is acquired through an external intelligent device. The self-adjustment module is configured to automatically adjust the initial bilateral stimulation parameters based on the real-time monitored emotional state and a preset adjustment strategy, thereby achieving adaptive adjustment of a personalized solution.

In an embodiment, the digital therapy processing system is deployed on an intelligent terminal, which can be a smartphone or tablet computer, among others, without limitation, and includes corresponding display screens, audio modules, and interfaces.

The digital therapy processing system is also connected to an external intelligent device to acquire the monitoring data, as well as to vibration devices to provide tactile stimulation to the user. In other words, the external intelligent device at least includes sensors for collecting physiological signals, imaging devices, and stimulation execution devices.

During operation, the self-assessment information is collected through voice interaction, which includes basic information, current emotional state, and description of traumatic memories of the user. The user is then guided to rate the intensity of emotions related to the traumatic memories on a scale of 0 to 10. The initial bilateral stimulation parameters are set based on this rating. During implementation, the initial bilateral stimulation parameters can also be set through user input.

The bilateral stimulation experience includes visual stimulation, tactile stimulation, and auditory stimulation.
a) Visual stimulation: a moving target is displayed on the screen, with adjustable speed (0.5-3 seconds per round trip).
b) Tactile stimulation: bilateral vibration is provided through an external vibration device, such as a wristband, with adjustable frequency of 1-10 hertz (Hz) and five intensity levels.
c) Auditory stimulation: alternating binaural sounds are provided through headphones, with a rhythm range of 60-150 beat per minute (bpm) and selectable sound types.

The digital therapy processing system also has a real-time emotional state monitoring mechanism, which specifically includes the follows.
1. An artificial intelligence technology is used, including: monitoring an emotional state of the user in real-time through multiple sensors.
2. Skin electrical signals are monitored, including: monitoring the skin conductance level (SCL) and skin conductance response (SCR) through external sensors.

During the operation, each of the visual stimulation, the tactile stimulation, and the auditory stimulation includes multiple sets of stimulation, and an interval time is preset between the visual stimulation and the tactile stimulation, between the visual stimulation and the auditory stimulation, and between the tactile stimulation and the auditory stimulation.

For example, the visual stimulation includes 6 sets, the tactile stimulation includes 4 sets, and the auditory stimulation includes 3 sets, there is a certain interval between each type of stimulation.

In an embodiment, to achieve diversity in stimulation and enhance the corresponding effects, for each of the visual stimulation, the tactile stimulation, and the auditory stimulation, an execution content of each stimulation is different from one another. For example, the first set of the visual stimulation displays the moving target on the screen along a regular linear trajectory, while the second set of the visual stimulation displays the moving target along an irregular linear trajectory. Other types of stimulation can be customized to have different execution content, and there is no limitation or further elaboration on this.

Furthermore, to enhance the accuracy of the adjustment, in another embodiment, based on the above-mentioned technical solution, the digital therapy processing system also includes a baseline module. The baseline module is configured to perform a baseline test on the user before providing the comprehensive bilateral stimulation experience, to obtain personalized baseline values. The personalized baseline values are then transmitted to the self-adjustment module as a reference for subsequent automatic parameter adjustments.

During adaptive adjustment, the bilateral stimulation parameters are automatically adjusted based on the real-time monitored emotional state in combination with the preset adjustment strategy, which specifically includes the follows. When the monitoring data of the user is detected to be below a first threshold of the personalized baseline values of the user, which indicates the user being in a higher stress state. In response to the user being in the higher stress state, automatically reducing the initial bilateral stimulation parameters to lower an intensity of stimulation. Simultaneously, a duration of a single therapy is shortened. The lowering the intensity of stimulation includes reducing each type of stimulation by the same or different proportions. When the monitoring data of the user is detected to rise rapidly and exceed a second threshold of the personalized baseline values of the user, which indicates the user being in a highly tense state. In response to the user being in the highly tense state, the initial bilateral stimulation parameters are automatically adjusted to first suspend existing visual stimulation, and followed by switching to a milder stimulation of the auditory stimulation or the tactile stimulation. The first threshold and the second threshold are different from each other and can be set flexibly.

In an embodiment, the obtained monitoring data includes heart rate variability, skin conductance level, facial expressions, vocal characteristics, and body movements. By integrating multiple types of data, the digital therapy processing system can more accurately reflect the personalized characteristics of the user. Among them, the vocal characteristics include pitch variation, loudness, and speech rate; and the body movements include movement amplitude and movement frequency. Scores are then assigned to each corresponding sub-item.

The heart rate variability (HRV) and the skin conductance level (SCL) are taken as two core physiological indicators as examples for illustration. The two indicators can relatively objectively reflect the emotional arousal state of the user and have the characteristics of real-time and measurability.

The digital therapy processing system will perform a baseline test lasting 3-5 minutes before the formal start of the treatment (that is, before providing the comprehensive bilateral stimulation experience), to record baseline values of HRV and SCL of the user in a calm state. The personalized baseline values obtained will serve as an important reference point for subsequent parameter adjustments.

HRV is taken as an example, when the HRV value of the user is detected to be 20% lower than the personal baseline value of the user, it indicates that the user is in a higher stress state. At this time, the system will automatically reduce the stimulation intensity. Specifically, this is manifested as slowing down the movement speed of the visual stimulation (gradually reducing from the personal baseline speed to about three times slower), reducing the tactile vibration frequency (to one-third of the baseline frequency), and slowing down the audio rhythm (to two-thirds of the baseline rhythm). Meanwhile, the digital therapy processing system will shorten the duration of a single therapy, controlling it within 20-30 minutes.

For the SCL indicator, the digital therapy processing system records the baseline value of the user in a calm state and uses it as a reference standard. When the SCL rises rapidly and exceeds five times the personal baseline value, it indicates that the user is in a highly tense state. In this case, the digital therapy processing system will first suspend the current visual stimulation, as the visual channel may exacerbate tension of the user. Instead, it switches to a milder auditory or tactile stimulation mode. Specifically, the digital therapy processing system will recommend using soothing background music (with a rhythm close to the baseline heart rate of the user) or a gentle tactile vibration mode (at half the baseline frequency).

In an embodiment, during implementation, when certain predefined specific conditions are met, the digital therapy processing system will proactively suggest switching the stimulation method.

Specifically, it is recommended to switch from the visual stimulation to the auditory stimulation or the tactile stimulation when the following conditions are detected:
when the HRV value remains low (below 40% of the personal baseline value) and the SCL value stays at a high level (more than three times the personal baseline value) for over five minutes;
when the user reports or the digital therapy processing system detects obvious symptoms of eye fatigue; and
when in a highly stressed state (HRV value below 60% of the personal baseline value).

On the contrary, if the user is in a low-activation state (the HRV value close to or higher than the personal baseline value, and the SCL value within one to two times the baseline value), the digital therapy processing system will suggest appropriately increasing the stimulation intensity or combining multiple stimulation methods to enhance the therapeutic effect. It should be noted that such adjustments always follow the principle of gradual progression to avoid discomfort that may be caused by sudden changes.

In an embodiment, the digital therapy processing system further includes a storage module, and the storage module is configured to record data during a therapeutic process and to encrypt storage and access control to ensure data security. Specifically, encrypted storage includes: encrypting and storing the treatment data on a local device or a secure cloud server. Access control includes: employing dual-factor authentication to ensure data security.

The recorded data includes: automatically recorded progress of each treatment session, including the content of traumatic memories, changes in emotional ratings, and the stimulation parameters used.

By securely storing the data of the entire process for subsequent analysis and reference in treatment.

The digital therapy processing system based on multimodal and intelligent monitoring provided in the embodiment of the disclosure has the following advantages as follows. By combining EMDR with multimodal and intelligent monitoring; while initiating multimodal bilateral stimulation, real-time emotional state monitoring is conducted at the same time. Based on the monitoring results, the digital therapy processing system automatically adjusts the bilateral stimulation parameters to achieve adaptive adjustment of the personalized solution, to ensure safety and effectiveness of the entire process, thereby reducing reliance on professional therapists, enhancing EMDR accessibility. Therapy effectiveness is enhanced through multimodal stimulation, to provide a more comprehensive sensory experience. The real-time emotional state monitoring is used to make timely adjustments to ensure the safety of treatment. Its automated operation has improved convenience and is suitable for home-based self-treatment, and it can perform adaptive adjustments of the personalized solution based on monitoring conditions to meet the usage needs of different users.

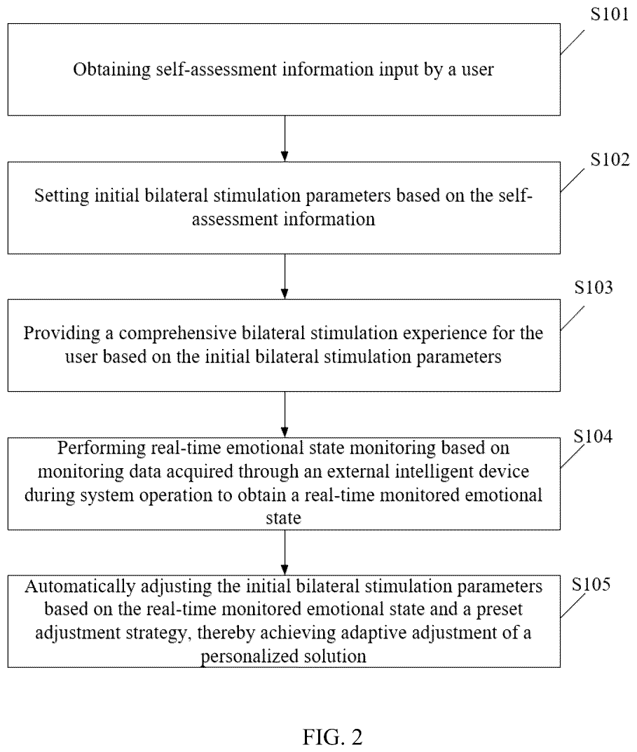

Correspondingly, based on the same inventive concept, the disclosure also provides a digital therapy processing method based on multimodal and intelligent monitoring. As shown in FIG. 2, which illustrates a flowchart of the digital therapy processing method based on multimodal and intelligent monitoring. Since the method is essentially similar to the system in the embodiment, the description is relatively simplified. For details, reference can be made to the corresponding parts of description of the system. The following description of the method is merely illustrative.

As shown in FIG. 2, a digital therapy processing method based on multimodal and intelligent monitoring provided in the embodiment of the disclosure is illustrated. The method is applied to the digital therapy processing system based on multimodal and intelligent monitoring described earlier. The method includes steps as follow.
S101: self-assessment information input by a user is obtained;
S102: initial bilateral stimulation parameters are set based on the self-assessment information;
S103: a comprehensive bilateral stimulation experience is provided based on the initial bilateral stimulation parameters, and the bilateral stimulation experience includes visual stimulation, tactile stimulation, and auditory stimulation;
S104: real-time emotional state monitoring is performed based on obtained monitoring data during system operation to obtain a real-time monitored emotional state of the user, and the monitoring data is acquired through an external intelligent device; and
S105: the initial bilateral stimulation parameters are automatically adjusted based on the real-time monitored emotional state and a preset adjustment strategy, thereby achieving adaptive adjustment of a personalized solution.

In an embodiment, each of the visual stimulation, the tactile stimulation, and the auditory stimulation includes multiple sets of stimulation, and an interval time is preset between the visual stimulation and the tactile stimulation, between the visual stimulation and the auditory stimulation, and between the tactile stimulation and the auditory stimulation. For each of the visual stimulation, the tactile stimulation, and the auditory stimulation, an execution content of each of the plurality of sets of stimulation is different from one another.

The digital therapy processing method further includes step as follows.

A baseline test is performed on the user before providing the comprehensive bilateral stimulation experience, to obtain personalized baseline values, and followed by transmitting the personalized baseline values to the self-adjustment module as a reference for subsequent automatic parameter adjustment.

In an embodiment, the automatically adjusting the initial bilateral stimulation parameters based on the real-time monitored emotional state and a preset adjustment strategy includes steps as follows:

When the monitoring data of the user is detected to be below a first threshold of the personalized baseline values of the user, which indicates the user being in a higher stress state. In response to the user being in the higher stress state, automatically reducing the initial bilateral stimulation parameters to lower an intensity of stimulation. And a duration of a single therapy simultaneously.

When the monitoring data of the user is detected to rise rapidly and exceed a second threshold of the personalized baseline values of the user, which indicates the user being in a highly tense state. In response to the user being in the highly tense state, the initial bilateral stimulation parameters are automatically adjusted to first suspend existing visual stimulation, and followed by switching to a milder stimulation of the auditory stimulation or the tactile stimulation.

The digital therapy processing method based on multimodal and intelligent monitoring provided in the embodiment of the disclosure is based on the same inventive concept as the digital therapy processing system based on multimodal and intelligent monitoring described earlier, and it achieves the same beneficial effects. Therefore, further elaboration is omitted here.

Finally, it should be noted that the above embodiments are merely used to illustrate the technical solutions of the disclosure and are not intended to limit it. Although the disclosure has been described in detail with reference to the foregoing embodiments, those skill in the art should understand that they can still modify the technical solutions described in the foregoing embodiments or replace some or all of the technical features with equivalent ones. These modifications or replacements do not cause the essence of the corresponding technical solutions to depart from the scope of the technical solutions of the embodiments of the disclosure. They should all be covered within the scope of the claims and specification of the disclosure.

What is claimed is:

1. A digital therapy processing system based on multimodal and intelligent monitoring, applied in an intelligent terminal, wherein the system comprises:
a data acquisition module, configured to obtain self-assessment information input by a user;
a setting module, configured to set initial bilateral stimulation parameters based on the self-assessment information;
a multimodal bilateral stimulation module, configured to provide a comprehensive bilateral stimulation experience for the user based on the initial bilateral stimulation parameters, wherein the bilateral stimulation experience comprises visual stimulation, tactile stimulation, and auditory stimulation;
a monitoring module, configured to perform real-time emotional state monitoring based on obtained monitoring data during system operation to obtain a real-time monitored emotional state of the user; wherein the monitoring data is acquired through an external intelligent device; and
a self-adjustment module, configured to automatically adjust the initial bilateral stimulation parameters based on the real-time monitored emotional state and a preset adjustment strategy, thereby achieving adaptive adjustment of a personalized solution.

2. The digital therapy processing system as claimed in claim 1, further comprising:
a storage module, configured to record data during a therapeutic process, and configured to encrypt storage and access control to ensure data security.

3. The digital therapy processing system as claimed in claim 1, wherein each of the visual stimulation, the tactile stimulation, and the auditory stimulation comprises a plurality of sets of stimulation, and an interval time is preset between the visual stimulation and the tactile stimulation, between the visual stimulation and the auditory stimulation, and between the tactile stimulation and the auditory stimulation.

4. The digital therapy processing system as claimed in claim 3, wherein, for each of the visual stimulation, the tactile stimulation, and the auditory stimulation, an execution content of each of the plurality of sets of stimulation is different from one another.

5. The digital therapy processing system as claimed in claim 4, further comprising:
a baseline module, configured to perform a baseline test on the user before providing the comprehensive bilateral stimulation experience, to obtain personalized baseline values, and transmit the personalized baseline values to the self-adjustment module as a reference for subsequent automatic parameter adjustment.

6. The digital therapy processing system as claimed in claim 5, wherein the automatically adjust the initial bilateral stimulation parameters based on the real-time monitored emotional state and a preset adjustment strategy comprises:
when the monitoring data of the user is detected to be below a first threshold of the personalized baseline values of the user, indicating the user being in a stress state; in response to the user being in the stress state, automatically reducing the initial bilateral stimulation parameters to lower an intensity of stimulation; and shortening a duration of a single therapy simultaneously;
when the monitoring data of the user is detected to rise and exceed a second threshold of the personalized baseline values of the user, indicating the user being in a tense state; in response to the user being in the tense state, automatically adjusting the initial bilateral stimulation parameters to first suspend existing visual stimulation, and switching to the auditory stimulation or the tactile stimulation.

7. A digital therapy processing method based on multimodal and intelligent monitoring, applied to the digital therapy processing system as claimed in claim 1, wherein the method comprises:
obtaining self-assessment information input by a user;
setting initial bilateral stimulation parameters based on the self-assessment information;
providing a comprehensive bilateral stimulation experience for the user based on the initial bilateral stimulation parameters, wherein the bilateral stimulation experience comprises visual stimulation, tactile stimulation, and auditory stimulation;
performing real-time emotional state monitoring based on obtained monitoring data during system operation to obtain a real-time monitored emotional state of the user, wherein the monitoring data is acquired through an external intelligent device; and
automatically adjusting the initial bilateral stimulation parameters based on the real-time monitored emotional state and a preset adjustment strategy, thereby achieving adaptive adjustment of a personalized solution.

8. The digital therapy processing method as claimed in claim 7, wherein each of the visual stimulation, the tactile stimulation, and the auditory stimulation comprises a plurality of sets of stimulation, and an interval time is preset between the visual stimulation and the tactile stimulation, between the visual stimulation and the auditory stimulation, and between the tactile stimulation and the auditory stimulation; for each of the visual stimulation, the tactile stimulation, and the auditory stimulation, an execution content of each of the plurality of sets of stimulation is different from one another.

9. The digital therapy processing method as claimed in claim 7, comprising:
performing a baseline test on the user before providing the comprehensive bilateral stimulation experience, to obtain personalized baseline values, and transmitting the personalized baseline values to the self-adjustment module as a reference for subsequent automatic parameter adjustment.

10. The digital therapy processing method as claimed in claim 9, wherein the automatically adjusting the initial bilateral stimulation parameters based on the real-time monitored emotional state and a preset adjustment strategy comprises:
when the monitoring data of the user is detected to be below a first threshold of the personalized baseline values of the user, indicating the user being in a stress state; in response to the user being in the stress state, automatically reducing the initial bilateral stimulation parameters to lower an intensity of stimulation; and shortening a duration of a single therapy simultaneously;
when the monitoring data of the user is detected to rise and exceed a second threshold of the personalized baseline values of the user, indicating the user being in a tense state; in response to the user being in the tense state, automatically adjusting the initial bilateral stimulation parameters to first suspend existing visual stimulation, and switching to a the auditory stimulation or the tactile stimulation.
